(19) United States
(12) Patent Application Publication
(10) Pub. No.: US 2010/0316635 A1
(43) Pub. Date: Dec. 16, 2010

(54) KIT FOR DIAGNOSIS OF BREAST CANCER USING HERCEPTIN, A COMPOSITION COMPRISING HERCEPTIN AND A METHOD FOR DETECTING HERCEPTIN-SENSITIVE HER2 OVEREXPRESSED CELL USING THE SAME

(75) Inventors: Eun Sook Lee, Seoul (KR); Keon Wook Kang, Seoul (KR); Byong Chul Yoo, Goyang-si (KR); Ho-Young Lee, Seoul (KR); Sun Young Kong, Goyang-si (KR); Se Hun Kang, Goyang-si (KR); Nam Suk Baek, Changwon-si (KR); Bu-Mi Kwon, Incheon (KR); Young Mi Kwon, Goyang-si (KR)

Correspondence Address: SEED INTELLECTUAL PROPERTY LAW GROUP PLLC, 701 FIFTH AVE, SUITE 5400, SEATTLE, WA, 98104, US

(73) Assignee: NATIONAL CANCER CENTER, Goyang-si (KR)

(21) Appl. No.: 12/738,833

(22) PCT Filed: Oct. 16, 2008
(86) PCT No.: PCT/KR08/06127

§ 371 Date: Aug. 18, 2010

(30) Foreign Application Priority Data
Oct. 19, 2007 (KR) 10-2007-0105736

(51) Int. Cl. A61K 39/395; G01N 33/574; C07K 16/30; A61P 35/00

(52) U.S. Cl. 424/133.1; 435/7.23; 530/387.3

(57) ABSTRACT

A kit and a composition comprising Herceptin, an antibody binding specifically to HER2, based on detecting Herceptin-sensitive HER2-overexpressing cells are provided for diagnosing cancer. A method of detecting Herceptin-sensitive HER2-overexpressing cells using the same is also provided.

### TECHNICAL FIELD

The present invention relates to a kit and a composition for diagnosing cancer comprising Herceptin, an antibody binding specifically to HER2, based on detecting Herceptin-sensitive HER2-overexpressing cells. The present invention is also concerned with a method of detecting Herceptin-sensitive HER2-overexpressing cells using the same.

### BACKGROUND ART

Human epidermal growth factor receptor 2 (HER2) is a member of the epidermal growth factor receptor (EGFR) family of receptor tyrosine kinases, which constitute a signaling network that plays a critical role in proliferation and survival of breast carcinoma cells. The EGFR family is composed of erb1, erb2/HER2, erb3 and erb4, which modulate many normal cellular processes such as proliferation, survival, adhesion, migration and differentiation. Of the four ERB family members, erb2/HER2, as a ligand-less receptor, is known as the most potent oncoprotein implicated in breast cancer. HER2 is involved in normal growth and development of glandular tissue when expressed at normal levels, but abnormal overexpression or amplification of HER2 disrupts normal cellular modulation to promote an aggressive cancer phenotype in glandular tissue. This process is mediated by oligomerization of HER2 with other EGFR family members which in turn phosphorylates numerous downstream molecules and activates several signaling cascades. Of multiple cellular signaling pathways activated by HER2, SOS-the Ras-Raf-MEK-MAPK pathway, involved in cell proliferation, and the PI-3K/Akt pathway, involved in apoptosis, are representative mechanisms for cancer development. Preclinical and clinical studies revealed that HER2 overexpression is an important phenomenon that occurs from the early stage of oncogenesis, and plays in a critical role in cancer growth and progression. HER2 is overexpressed in about 20-30% of invasive breast cancer cases, and its overexpression is indicative of a more aggressive (malignant) cancer and is associated with poor prognosis.

Herceptin is a recombinant humanized monoclonal antibody that targets the extracellular domain of the HER2 protein. The binding of Herceptin to the extracellular domain of HER2 inhibits the heterodimerization of HER2 with other HER receptor family members, which is important in HER2-mediated signaling, and thus blocks the activation of downstream signaling cascades, resulting in decreased cell proliferation, increased apoptosis and decreased angiogenesis. For treatment with such Herceptin, HER2 overexpression must be confirmed in breast cancer.

HER2 is gaining great attention in breast cancer studies because HER2 gene amplification or protein overexpression has a prognostic value in breast cancer patients and a predictive value for response to treatment. Its prognostic value is still controversial, but HER gene amplification or protein overexpression has been reported to be indicative of poor prognosis and to be associated with significantly shorter survival. In particular, for treatment of metastatic or primary breast cancer patients with Herceptin as a monoclonal therapeutic agent against HER2/neu, HER2 amplification/overexpression is a decisive indicator. Such a value of HER2 was approved and recommended for use as a tumor marker for breast cancer by the American Society of Clinical Oncology (ASCO) in 2000. Most clinical practice guidelines recommend HER2 testing for all primary breast cancer patients. Thus, universal standardization of HER2 testing is necessary to achieve accurate HER2 status determination in breast cancer tissues.

HER2 status can be detected by assessing HER2 gene amplification using fluorescence in situ hybridization (FISH), chromogenic in situ hybridization (CISH) and polymerase chain reaction (PCR), or by analyzing increased HER2 mRNA transcripts using reverse transcription-polymerase chain reaction (RT-PCR), or by assessing HER2 protein overexpression using immunohistochemistry (IHC). IHC and FISH are the most commonly used techniques.

Immunohistochemistry, which is a technique routinely used in clinical diagnostics, has some advantages such as no need for additional equipment, short time of about 3 hrs for evaluation, relatively easy methodology and relatively low cost. However, there are disadvantages with the IHC assay: low detection sensitivity relative to other molecular genetic techniques, and varying results depending on the sensitivity and specificity of antibodies used to detect HER2, the use of antigen retrieval techniques and scoring. Also, since the representation of IHC results is highly subjective, it is prone to show interobserver variability even with the same technique such as the use of the same specimen or the same antibody. For example, according to the scoring guidelines of the FDA-approved IHC-based scoring system, HercepTest® (Dako), using a rabbit anti-HER2 polyclonal antibody as the primary antibody and a goat anti-rabbit immunoglobulin as the secondary antibody, a strong membrane staining is scored as 3+, and a weak to moderate membrane staining is graded as 2+. The determination of staining intensity to be strong or moderate is highly subjective and thus less objective. Indeed, using the same specimen, the agreement on 2+ IHC score was only 59% among pathologists. The HercepTest® system defines a grade of 2+ or higher as HER2/neu overexpression, but only about 25% of specimens with 2+ immunostaining scores exhibit HER2/neu gene amplification. Patients having apparent protein expression (IHC 2+) but not having gene amplification failed to show positive response to Herceptin treatment.

Fluorescence in situ hybridization (FISH) is the most accurate assay among currently available HER2/neu testing methodologies. The FISH assay has the highest detection sensitivity and rarely produces false-negative results. However, FISH has drawbacks in that it takes a relatively long time of about 2 days, requires the use of a fluorescent probe about 10 times more expensive than antibodies used in IHC, and does not directly detect cancer cells. Thus, tumor samples are primarily determined for HER2 status using IHC, and IHC 2+ samples should be retested with FISH. In many studies, the concordance between IHC 3+ and FISH positive was 95%, and IHC 3+ and FISH-positive patients had the identical response rates of 49% to Herceptin treatment. As noted above, IHC is typically performed first. Samples scored as 3+ (IHC 3+) are considered positive, IHC 0 or 1+ samples are considered negative, IHC 2+ samples are considered equivocal and retested with FISH for HER2 amplification.

Although candidates for Herceptin treatment are most suitably selected through IHC or FISH, the response rates to Herceptin treatment are merely less than 50%. This is because conventional HER2 testing detects HER2 amplification and overexpression in both Herceptin-sensitive and Herceptin-resistant cells and is thus difficult to identify a patient actually in need of Herceptin treatment. Thus, the development of a method capable of more suitably selecting a candidate for Herceptin treatment can reduce undesired wastes and side effects. Many efforts have been made so as to develop a method capable of standardizing and technically validating HER2 testing methodology.

### DISCLOSURE

### Technical Problem

In this regard, the present inventors conducted intensive and thorough research in order to overcome the limitation of conventional HER2 testing methods, and found that the use of Herceptin as a diagnostic antibody enables effective detection of HER2-overexpressed cells that are Herceptin-sensitive and thus respond to Herceptin treatment, other than cells merely overexpressing HER2, thereby leading to the present invention.

### Technical Solution

It is therefore an object of the present invention to provide a cancer diagnostic kit for detecting HER2-overexpressing cells being sensitive to Herceptin, the kit comprising HER2-specific Herceptin and detecting an HHR2 expression level in a sample through antigen-antibody complex formation.

It is another object of the present invention to provide a method of detecting HER2-overexpressing cells being sensitive to Herceptin using the diagnostic kit.

It is a further object of the present invention to provide a diagnostic composition for identifying cancer through detection of Herceptin-sensitive HER2-overexpressing cells, the composition comprising Herceptin.

### DESCRIPTION OF DRAWINGS

FIG. 1 shows a comparison of immunochemical staining results in the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistant), HCC70(HER2−) and MCF7(HER2−) when using an HER2 diagnostic kit of DAKO and when using Herceptin as a primary antibody.

FIG. 2 shows a comparison of immunochemical staining results in tissues from a breast cancer patient and a normal person, who are determined to be respectively positive and negative as measured by an HER2 diagnostic kit of DAKO in combination FISH, when using the HER2 diagnostic kit alone and when using Herceptin as a primary antibody.

FIG. 3 show a comparison of immunochemical staining results in the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistant), HCC70(HER2−) and MCF7(HER2−), all alive, using an HER2 diagnostic kit of DAKO in combination with Q-dot as a label and when using Herceptin conjugated with Q-dot.

FIG. 4 shows immunohistochemical staining results in cell blocks of the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistant), HCC70(HER2−) and MCF7(HER2−) when using Herceptin as a primary antibody and Q-dot conjugated anti-human immunoglobulin G as a secondary antibody after pretreatment with Proteinase K.

FIG. 5 shows immunohistochemical staining results in case of pretreatment with Pepsin.

### BEST MODE

The present invention pertains to a kit and a composition for diagnosing cancer comprising Herceptin, an antibody binding specifically to HER2, through detection of Herceptin-sensitive HER2-overexpressing cells. The present invention also relates to a method of detecting Herceptin-sensitive HER2-overexpressing cells using the same.

In accordance with an aspect thereof, the present invention provides a cancer diagnostic kit comprising Herceptin for detecting Herceptin-sensitive HER2-overexpressing cells by detecting an expression level of HER2 in a sample through antigen-antibody complex formation.

“Herceptin”, used in the present kit, is a humanized monoclonal antibody for treating breast cancer and is developed by and commercially available from Genentech (San Francisco, Calif., USA). It may be purchased from Genentech, or may be prepared using a known method.

The present kit may include Herceptin or an antibody fragment or a variant thereof.

The term “antibody fragment”, as used herein, indicates a portion of an antibody molecule, which preferably includes an antigen-binding site or a variable region thereof. Examples of antibody fragments include Fab, F(ab′), F(ab′)₂, and scFv. Such antibody fragments may be obtained using proteolytic enzymes. Papain digests a whole antibody into two identical antigen-binding fragments (Fab fragments), each of which has a single antigen-binding site, and Fc fragments. Pepsin is used to generate F(ab′)₂ fragments, which have an antigen-binding site and retain an ability to cross-link with its antigen. Fab fragments contain the variable domain of the light chain and the first constant domain (CH1) of the heavy chain. Fab′ fragments differ from the Fab fragments in terms of having the hinge region containing one or more cysteine residues at the C-terminus (carboxyl terminus) of the heavy chain CH1 domain. The term “variant”, as used herein, refers to an antibody molecule that retains biological activity (action or structural) substantially similar to that of the antibody molecule according to the present invention, Herceptin, that is, substantially similar substrate specificity or substrate cleavability. For example, it may include one or a few point mutations, substitution, deletion or insertion of one or a few nucleotides, or substitution, deletion or insertion of one or a few amino acids. The variant retains its biological activity such as antibody-binding activity, and has at least partially or even more enhanced biological activity.

The “antigen-antibody complex formation” for measurement of HER2 expression levels in samples according to the present invention may be detected using any method capable of measuring antigen binding to the present antibody. Such methods are well known in the art, preferably selected from the group consisting of immunohistochemistry, immunoblot, immunoprecipitation, enzyme-linked immunosorbent assay (ELISA), agglutination and radioimmunoassay. Immunohistochemical techniques are particularly preferred.

The term “Herceptin-sensitive HER2-overexpressing cells”, as used herein, indicates cells showing therapeutic response to Herceptin among cells overexpressing HER2 on the cell surface relative to normal cells. HER2 is a 185-kD transmembrane glycoprotein that is encoded by a proto-oncogene located on the long arm of chromosome 17 (17q21). Since the cytoplasmic domain of p185 has tyrosine kinase activity, the structure contains a cytoplasmic domain having a homology of 40% and an extracellular domain having a homology of 85% with epidermal growth factor receptor. HER2 expression is found in breast cancer, lung cancer, ovarian cancer, stomach cancer, and the like. Among various breast carcinoma cell lines established up to date, herceptin-sensitive HER2-overexpressing cells include AU565, SK-BR-3, SK-BR-3 and HCC1569 cells. The kit according to the present invention does not detect Herceptin-resistant cells, such as JIMT-1 cells, which overexpress HER2 but do not respond to Herceptin treatment, but detects only the aforementioned Herceptin-sensitive cells.

The cancer diagnostic kit of the present invention enables the detection of cancer caused by HER2 overexpression. Examples of HER2-overexpressing cancer include, but are not limited to, breast cancer, lung cancer, ovarian cancer, and stomach cancer. In particular, the kit is preferably used to detect breast cancer.

The diagnostic kit may further include a secondary antibody conjugated with a label, which is detected through a colorimetric reaction with a substrate, a color development substrate solution for the colorimetric reaction with the label, and a washing solution to be used in each reaction step.

As the label to be conjugated to a secondary antibody, a commonly used label detected through a colorimetric reaction is preferred. Examples of detectable labels include Quantum dots (Q-dots), Horseradish peroxidase (HRP), alkaline phosphatase, glucose oxidase, luciferase, β-D-galactosidase, malate dehydrogenase (MDH), acetylcholinesterase, colloid gold, fluoresceins, radioisotopes, and dyes. Fluorescent substances include fluoresceine isothiocyanate and phycobili proteins. Luminescent substances include isolucinol and lucigenin. Radioactive substances include ¹²⁵I, ¹³¹I, ¹⁴C and ³H. In addition to those described above, any one that can be used in immunological analysis may be used. Particularly preferably, Q-dots, as described in Example 3, enable more accurate measurement for the degree of color development. The present invention employs a goat anti-human IgG-HRP conjugate (Zymed) and a Q-dot-antibody conjugate.

A color development substrate is preferably determined depending on a label detected through colorimetric reaction and may be exemplified by DAB (diaminobenzidine), AEC (3-amino-9-ethylcarbasole), BCIP/NBT (5-bromo-4-chloro-3-indolyl-phosphate/nitroblue tetrazolium), BCIP/INT (5-bromo-4-chloro-3-indolyl phosphate/iodonitrotetrazolium), NF (New fuchsin), FRT (Fast Red TR Salt), TMB (3,3′,5,5′-tetramethyl benzidine), ABTS [2,2′-azino-bis(3-ethylbenzothiazoline-6-sulfonic acid)] and OPD (o-phenylenediamine). The color development substrate TMB is degraded by HRP, used as a label of a secondary antibody conjugate, to form a chromogen. Visualization of the chromogen is an indication of the presence of an HCCR-1 protein antigen. In the present invention, a detection system provided with HRP (Horseradish peroxidase) was used in combination with DAB (diaminobenzidine) as a chromogenic substrate.

As the washing solution, distilled water, TBS (Tris buffered saline) Tween buffered saline, PBS (phosphate buffered saline), PSS, NaCl and Tween 20 may be used according to stages. After an antigen-antibody complex is reacted with a secondary antibody, the resulting conjugate is washed three to six times with the washing solution in a reactor.

In accordance with another aspect thereof, the present invention provides a method for detecting HER2-overexpressing cells sensitive to Herceptin using the diagnostic kit. In greater detail, the present invention provides a method for detecting HER2-overexpressing cells sensitive to Herceptin, comprising: 1) obtaining a specimen from a subject; 2) treating the specimen with protease; and 3) treating the specimen with Herceptin to form an antigen-antibody complex.

As used herein, the term “specimen from a subject” indicates cells or a biopsy isolated from a subject to determine whether the subject is affected by HER2 overexpression-induced cancer or whether the subject is at risk of being affected by such cancer. The specimen may be isolated from a subject with cancer (e.g., breast cancer tissue) or a healthy person. The specimen may be processed into, for example, paraffin-embedded tissues, frozen tissues, formalin fixed tissues, or cell smear. Of them, paraffin-embedded tissues are most widely used. The fixation of a specimen is the most important for morphological observation of cells or tissues. In suitably fixed tissues, cell organelles are well maintained. Insufficient fixation makes cell organelles unstable in morphological structure. Excessive fixation causes antigen loss and non-specific responses. Tissue fixatives may be divided into coagulation-type fixatives and non-coagulation (denaturation) type fixatives. Formalin, a most widely used fixative, is of non-coagulation type. While infiltrating tissues at a rate of 1 mm per hour, formalin fixes the tissues. Formalin destroys epitopes only to a small extent and crosslinks proteins and peptides, thereby maintaining the morphology of cells.

Between the step 1) and the step 3), the specimen obtained in the step 1) is pre-treated with proteinase to retrieve antigenicity.

By the term “retrieval of antigenicity”, or corresponding phrases, as used herein, it is meant that a crosslink between an epitope and a protein in the paraffin embedded tissue fixed by formalin is broken to cause an antigen to be readily detected. Recently, when an antigen is difficult for conventional staining methods to detect from a paraffin embedded tissue fixed by formalin, an antigen retrieval system based on microwave, autoclave or a pressure cooker has been used. Also, the crosslinks formed by formalin fixation may be broken by use of proteinase such as pepsin, rennin, trypsin, chymotrypsin, cathepsin, papain, ficin, thrombin, renin, collagenase, bromelain and bacterioproteinase, peptidase, proteinase A, or proteinase K. Preferable is proteinase K or pepsin.

In a preferred example of the present invention, good retrieval of antigenicity could be obtained by pre-incubating paraffin-embedded tissue slides at 37° C. for 3 min with proteinase K (0.02 mg/ml). Proteinase K (Tritirachium alkaline proteinase), discovered in extracts of Tritirachium album Limber, is a kind of serine endopeptidase which rapidly digests proteins and retains its activity even in the presence of a detergent such as urea or sodium dodecyl sulfate (SDS). Further, proteinase K is used for the isolation of DNA or RNA thanks to its ability to digest keratin. Proteinase K may be commercially available as an aqueous solution, a suspension, or a freeze-dried powder (for example, a product from Invitrogen).

Treatment with Herceptin to form an antigen-antibody complex is carried out ex vivo. This antigen-antibody binding may be analyzed using a well-known technique, examples of which include, but are not limited to, immunohistochemistry, immunoblot, immunoprecipitation, ELISA (enzyme linked immunosorbent assay), agglutination and radio-immuno assay, with preference for immunohistochemical techniques. Various modifications and applications may be possible in the techniques.

In an embodiment, the method of the present invention may further comprise detecting the antigen-antibody complex of step 2) by use of a label-conjugated secondary antibody and a color development solution.

Descriptions for the label, the secondary antibody and the color development solution useful in the diagnostic method of the present invention may refer to those in the diagnostic kit.

In accordance with a further aspect thereof, the present invention provides a pharmaceutical composition for the diagnosis of cancer, based on detecting Herceptin-sensitive HER2-overexpressing cells, comprising Herceptin.

The term “pharmaceutical composition”, as used herein, is intended to refer to a composition comprising Herceptin as an active ingredient. The composition may further comprise pharmaceutically acceptable carriers or excipients. The pharmaceutical composition aims to make it easy to administer the active ingredient. The term “active ingredient” as used herein indicates a peptide or an antibody agent providing a desired biological effect. The term “pharmaceutically acceptable carrier” as used herein means a vehicle or diluent which aids the administration of the active ingredient with neither stimulating the subject nor inhibiting biological activities and properties of the active ingredient. An auxiliary agent may be included within the scope of the pharmaceutically acceptable carrier. Examples of suitable carriers include, but are not limited to, soluble carriers, e.g., well-known physiologically acceptable buffers (PBS, etc.), insoluble carriers, e.g., metal-coated polymer, such as polystyrene, polyethylene, polypropylene, polyester, polyacrylonitril, fluorine resins, crosslinkable dextran, polysaccharides, latex, etc., paper, glass, metal, agarose and combinations thereof. The term “excipient”, as used herein, means an inert material which makes it easy to administer the active ingredient. Examples of the excipient useful in the present invention include calcium carbonate, calcium phosphate, various saccharides, cellulose derivatives, gelatin, vegetable oil and polyethylene glycol.

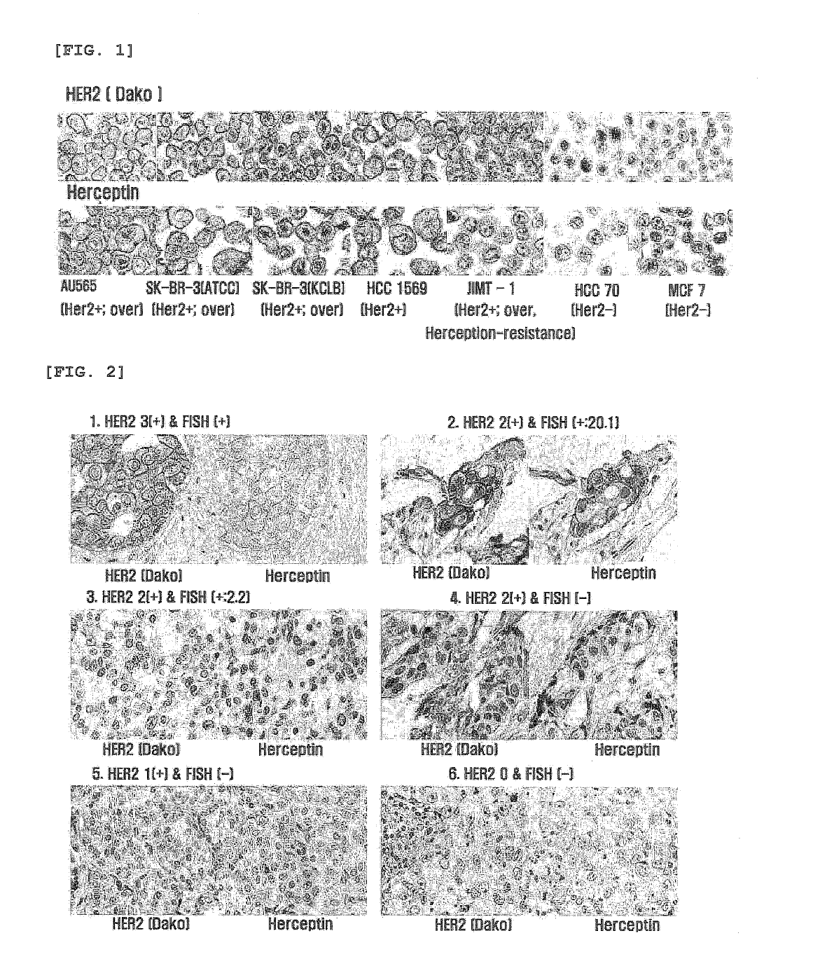

Conventional HER2 assay kits, such as that commercially available from DAKO, strongly stain even Herceptin-resistant, HER2-overexpresing cells such as JIMT-1, making it difficult to identify a patient actually in need of Herceptin treatment (FIGS. 1 and 3). In contrast, Q dot-Trastuzumab in accordance with the present invention Herceptin-sensitive reads only HER2-overexpressing cell lines, thereby accurately identifying a patient in need of Herceptin treatment (FIGS. 1 to 4).

### MODE FOR INVENTION

A better understanding of the present invention may be obtained through the following examples which are set forth to illustrate, but are not to be construed as the limit of the present invention.

### Example 1

### Immunohistochemical Staining with HER2 Diagnostic Kit of DAKO

Among the breast cancer cell lines established thus far, the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistance), HCC70(HER2−) and MCF7(HER2−) were selected. Each cell line was cultured at a viability of 90% in a 75 cm² flask under a 37° C., 5% CO₂ condition for 5 days with provision of SK-BR-3 and JIMT-1 cells with DMEM (Dulbecco's Modified Eagle's Medium, Gibco) supplemented with 10% heat-inactivated FBS (Gibco), 100 μunits/100 μg penicillin/streptomycin (Gibco), and AU565, HCC 1569, MCF 7 and HCC 70 cells with RPMI 1640 (Gibco) supplemented with 10% heat-inactivated FBS (Gibco), 100 μunits/100 μg penicillin/streptomycin (Gibco).

Each of the cell lines cultured was embedded in paraffin block which was then sectioned into 4 μm-thick slices. Likewise, cancer tissues excised from breast cancer patients were also embedded in paraffin blocks and sectioned into 4 μm-thick slices. These slices were attached onto slides and immersed for 5 min in xylene (twice) to remove paraffin. The slices were rehydrated by immersion for 3 min in 100% ethanol (twice) and for 1 min in 95% ethanol (once) and washed for 1 min with distilled water (five times). While being immersed in citrate buffer (pH 6.0), the slides were treated for 20 min with microwaves to retrieve antigenicity. In order to remove endogenous peroxidase activity therefrom, the slides were immersed for 10 min in a hydrogen peroxide blocking buffer (Labvision), washed for 4 min with TBS Tween buffer, and treated for 10 min with the blocking solution provided for the Cap-Plus™diagnostic kit (Zymed, San Francisco Calif. USA). Treatment for 90 min with a 1:500 dilution of a primary antibody (polyclonal rabbit anti-human c-erbB-2, Dako) was followed by washing for 4 min with TBS tween buffer. Then, a secondary antibody was applied for 20 min to the slides which were then washed with TBS tween buffer and treated at room temperature with streptavidin-HRP. Colors were developed with DAB (diaminobenzidine) under a microscope. The specimens were counterstained with Mayer's hematoxylin, and mounted with a mounting medium before observation under an optical microscope.

All of the HER2-overexpressing cells AU565, SK-BR-3, SK-BR-3, HCC1569 and JIMT-1 were observed to be intensively stained at cell membranes. No stained cell membranes were found in HCC70 and MCF7, neither of which expresses HER2 (FIG. 1).

### Example 2

### Immunohistochemical Staining with Herceptin

Among the breast cancer cell lines established thus far, the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistance), HCC70(HER2−) and MCF7(HER2−) were selected. Each cell line was cultured at a viability of 90% in a 75 cm² flask under a 37° C., 5% CO₂ condition for 5 days with provision of SK-BR-3 and JIMT-1 cells with DMEM (Dulbecco's Modified Eagle's Medium, Gibco) supplemented 10% heat-inactivated FBS (Gibco), 100 μunits/100 μg penicillin/streptomycin (Gibco), and AU565, HCC 1569, MCF 7 and HCC 70 cells with RPMI 1640 (Gibco) supplemented with 10% heat-inactivated FBS (Gibco), 100 μunits/100 μg penicillin/streptomycin (Gibco).

Each of the cell lines cultured was embedded in paraffin block which was then sectioned into 4 μm-thick slices. Likewise, cancer tissues excised from breast cancer patients were also embedded in paraffin blocks and sectioned into 4 μm-thick slices. These slices were attached onto slides and immersed for 5 min in xylene (twice) to remove paraffin. The slices were rehydrated by immersion for 3 min in 100% ethanol (twice) and for 1 min in 95% ethanol (once) and washed for 1 min with distilled water (five times). In order to retrieve antigenicity, Proteinase K (0.02 mg/ml) was applied at 37° C. for 4 min to the cell block slides and at 37° C. for 30 min to the tissue block slides. The slides were immersed for 10 min in a hydrogen peroxide blocking buffer (Dako) to remove endogenous peroxidase activity therefrom and washed for 4 min with TBS Tween buffer. Afterwards, the slides were reacted for 5 min in Super Block (Scytek, Logan, Utah, USA) containing a blocking antibody to exclude non-specific immune responses, followed by washing with TBS tween buffer. The slides were then treated for 60 min with human-to-human block (Scytek) and washed with TBS tween buffer. Treatment for 90 min with a primary antibody (10 μg/ml, Herceptin, Genentech, San Francisco, Calif.) was followed by washing with TBS tween buffer. Then, a secondary antibody (HRP-Goat Anti-Human IgG conjugate, Zymed) was applied for 20 min to the slides which were then washed with TBS. Colors were developed with DAB (diaminobenzidine) under a microscope. The specimens were counterstained with Mayer's hematoxylin, and mounted with a mounting medium before observation under an optical microscope.

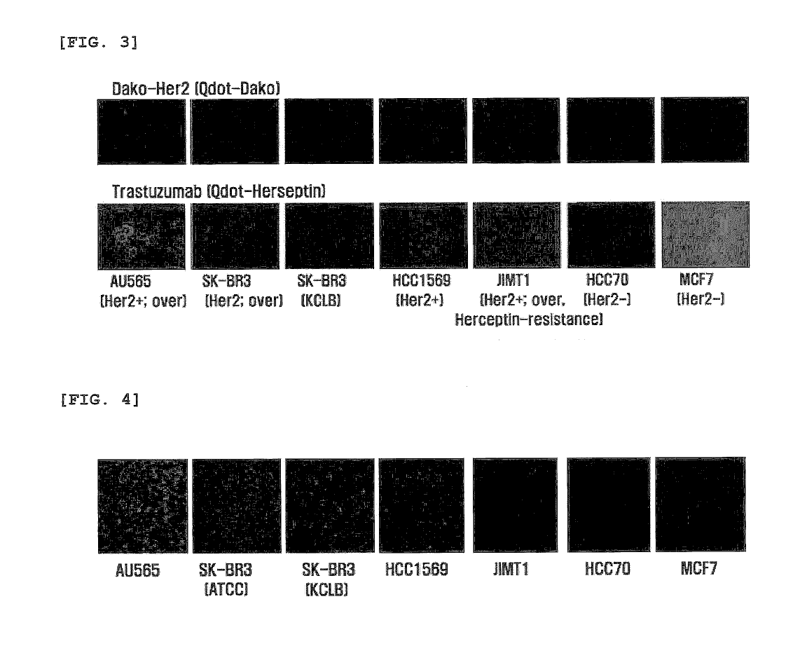

The HER2-overexpressing cells AU565, SK-BR-3, SK-BR-3 and HCC1569, which are all sensitive to Herceptin, were observed to be intensively stained at cell membranes. In contrast, no stained cell membranes were found in the HER2-overexpressing cell line JIMT-1, which is resistant to Herceptin, as well as in HCC70 and MCF7, neither of which expresses HER2 (FIG. 1). Further, the HER2 diagnostic kit from DAKO was observed to stain the tissues not only from breast cancer patients, but also from normal persons who were found to be negative as measured by FISH. In contrast, the immunohistochemical staining with Herceptin serving as a primary antibody was perfectly concordant with the records of FISH (FIG. 2).

Conventional HER2 testing with, for example, a kit from DAKO exhibits membrane staining in all HER-overexpressing cells, whether sensitive or resistant to Herceptin. The use of Herceptin as a diagnostic antibody in accordance with the present invention allows the selection of only Herceptin-sensitive cells from among HER2-overexpressing cells.

### Example 3

### Immunohistochemical Staining with Herceptin and Q-Dot

Among the breast cancer cell lines established thus far, the HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistance), HCC70(HER2−) and MCF7(HER2−) were selected. Each cell line was cultured at a viability of 90% in a 75 cm² flask under a 37° C., 5% CO₂ condition for 5 days with provision of SK-BR-3 and JIMT-1 cells with DMEM (Dulbecco's Modified Eagle's Medium, Gibco) supplemented with 10% heat-inactivated FBS (Gibco), 100 μunits/100 μg penicillin/streptomycin (Gibco), and AU565, HCC 1569, MCF 7 and HCC 70 cells with RPMI 1640 (Gibco) supplemented with 10% heat-inactivated FBS (Gibco), 100 μunits/100 μg penicillin/streptomycin (Gibco).

Each of the cell lines cultured was embedded in paraffin block which was then sectioned into 4 μm-thick slices. Likewise, cancer tissues excised from breast cancer patients were also embedded in paraffin blocks and sectioned into 4 μm-thick slices. These slices were attached onto slides and immersed for 5 min in xylene (twice) to remove paraffin. The slices were rehydrated by immersion for 3 min in 100% ethanol (twice) and for 1 min in 95% ethanol (once) and washed for 1 min with distilled water (five times). In order to retrieve antigenicity, Proteinase K (0.02 mg/ml) was applied at 37° C. for 4 min to the cell block slides and at 37° C. for 30 min to the tissue block slides. The slides were immersed for 10 min in a hydrogen peroxide blocking buffer (Dako) to remove endogenous peroxidase activity therefrom and washed for 4 min with TBS Tween buffer. Afterwards, the slides were reacted for 5 min in Super Block (Scytek, Logan, Utah, USA) containing a blocking antibody to exclude non-specific immune responses, followed by washing with TBS tween buffer. The slides were then treated for 60 min with human-to-human block (Scytek) and washed with TBS tween buffer. Treatment for 60 min with a primary antibody (10 μg/ml, Herceptin, Genentech, San Francisco, Calif.) was followed by washing with TBS tween buffer. Then, a Q-dot-conjugated secondary antibody (Q-dot 605 goat Anti-Human IgG conjugate, Invitrogen) was applied for 30 min to the slides which were then washed with TBS. Observation was performed under a fluorescence microscope.

As shown in FIG. 3, immunohistochemical staining with the HER2 diagnostic kit of DAKO in combination with Q-dot as a label for the antibody developed colors in all of the HER2-overexpressing cells AU565, SK-BR-3 (ATCC), SK-BR-3 (KCLB), HCC1569 and JIMT-1 whereas immunohistochemical staining with Q-dot-labeled Herceptin did not make color development in the Herceptin-resistant, HER2-overexpressing cell line JIMT-1 as well as in HCC70 and MCF7, neither of which expresses HER2 (FIG. 3). Accordingly, it was confirmed that the use of Herceptin as a diagnostic antibody allows the selection of only Herceptin-sensitive cells from among HER2-overexpressing cells. Q-dot was found to more accurately detect the target cells than in FIG. 1 or 2.

### Example 4

### Immunochemical Staining Using Proteinase K and Pepsin

The HER2/neu cell lines AU565(HER2+; over), SK-BR-3(ATCC-HER2+; over), SK-BR-3(KCLB-HER2+; over), HCC1569(HER2+; over), JIMT-1(HER2+; over, Herceptin-resistance), HCC70(HER2−) and MCF7(HER2−) were embedded in paraffin blocks which were then sectioned into 4 μm-thick slices. These slices were attached onto slides and immersed for 5 min in xylene (twice) to remove paraffin. The slices were rehydrated by immersion for 3 min in 100% ethanol (twice), for 1 min in 95% ethanol, for 1 min in 80% ethanol and for 1 min in 70% ethanol, and washed for 1 min with distilled water (five times).

In order to retrieve antigenicity, Proteinase K (0.02 mg/ml) was applied at 37° C. for 4 min to some of the slides while pepsin was applied for 4 min to the other slides. These slides were immersed for 20 min in 2% BSA/PBS and washed with PBS. Afterwards, the slides were reacted for 5 min in Super Block (Scytek, Logan, Utah, USA) containing a blocking antibody to exclude non-specific immune responses, followed by washing with PBS. The slides were then treated for 60 min with human-to-human block (Scytek) and washed with PBS. Treatment at room temperature for 90 min with a primary antibody (10 μg/ml, Herceptin, Genentech, San Francisco, Calif.) or for 60 min with the primary antibody in combination with Q-dot was followed by washing with PBS.

For typical immunohistochemical staining, the specimens were incubated at room temperature for 30 min with a secondary antibody (HRP-Goat Anti-Human IgG conjugate, Zymed). Colors were developed under a microscope. The slides were counterstained and mounted with a mounting medium before observation under an optical microscope.

As for immunohistochemical staining with Q-dot, the specimens were incubated for 30 min with a Q-dot-conjugated secondary antibody (Q-dot 605 goat Anti-Human IgG conjugate, Invitrogen) and washed with TBS before observation under a fluorescence microscope.

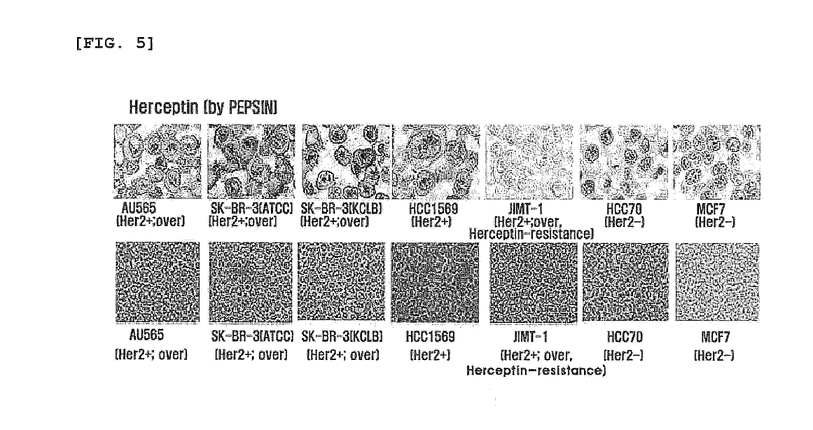

Immunohistochemical staining with Q-dot-labeled herceptin were found to develop colors in the Herceptin-sensitive, HER2-overexpresing cells AU565, SK-BR-3 (ATCC), SK-BR-3 (KCLB) and HCC1569, but not in the Herceptin-resistant, HER2-overexpressing cell JIMT-1 as well as in HCC70 and MCF7, neither of which expresses HER2 (FIGS. 4 and 5), whether they were pretreated with Proteinase K or pepsin. That is, as long as it is sensitive to Herceptin, an HER2-overexpressing cell line can be accurately detected by the method of the present invention irrespective of treatment with Proteinase K or pepsin. Accordingly, it was confirmed that the use of Herceptin as a diagnostic antibody allows the selection of only Herceptin-sensitive cells from among HER2-overexpressing cells.

### INDUSTRIAL APPLICABILITY

On the basis of the use of Herceptin as an antibody in the diagnosis of cancer caused by HER overexpression, as described hitherto, the present invention can fill the gap in detection ability of conventional diagnostic kits, that is, can determine whether HER2-overexpresing cells are sensitive or resistant to Herceptin, thereby accurately selecting patients in need of Herceptin treatment.

1. A kit, comprising Herceptin sensitive to HER2, for diagnosing cancer by detecting detecting Herceptin-sensitive, HER2-overexpressing cells through antigen-antibody complex formation.

2. The kit according to claim 1, wherein the cancer is breast cancer.

3. The kit according to claim 1, wherein the antigen-antibody complex formation is analyzed using a method selected from among immunohistochemical techniques, immunoblot, immunoprecipitation, ELISA (enzyme linked immunosorbent assay), agglutination and radio-immuno assay.

4. The kit according to claim 1, wherein the antigen-antibody complex formation is analyzed using an immunohistochemical technique.

5. The kit according to claim 1, further comprising a secondary antibody conjugated with a label, a chromogenic substrate which reacts with the label to develop a color, and washing solutions to be used according to reaction stages.

6. The kit according to claim 5, wherein the label of the secondary antibody is selected from a group consisting of Q dot (Quantum dot), HRP (Horseradish peroxidase), alkaline phosphatase, glucose oxidase, luciferase, β-D-galactosidase, MDH (malate dehydrogenase), acetylcholinesterase, colloidal gold, fluorescein, radioactive materials and dye.

7. The kit according to claim 5, wherein the label of the secondary antibody is Q dot (Quantum dot).

8. The kit according to claim 5, wherein the chromogenic substrate is selected from a group consisting of DAB (diaminobenzidine), AEC(3-amino-9-ethylcarbasole), BCIP/NBT (5-bromo-4-chloro-3-indolyl-phosphate/nitroblue tetrazolium), BCIP/INT (5-bromo-4-chloro-3-indolyl phosphate/iodonitrotetrazolium), NF (New fuchsin), FRT (Fast Red TR Salt), TMB (3,3′,5,5′-tetramethyl benzidine), ABTS [2,2′-azino-bis(3-ethylbenzothiazoline-6-sulfonic acid)] and OPD (o-phenylenediamine).

9. A method for detecting Herceptin-sensitive, HER2-overexpressing cells, comprising:
1) obtaining a specimen from a subject;
2) treating the specimen with proteinase; and
3) reacting the specimen with Herceptin to form an antigen-antibody complex.

10. The method according to claim 9, wherein the specimen is a tissue specimen from a breast cancer patient.

11. The method according to claim 9, wherein the proteinase is Proteinase K or pepsin.

12. The method according to claim 9, wherein the antigen-antibody complex is analyzed using a technology selected from a group consisting of immunohistochemical techniques, immunoblot, immunoprecipitation, ELISA (enzyme linked immunosorbent assay), agglutination) and radio-immuno assay.

13. The method according to claim 9, wherein the antigen-antibody complex is analyzed using an immunohistochemical technique.

14. The method according to claim 9, further comprising detecting the antigen-antibody complex by use of a secondary antibody conjugated with a label and a chromogenic substrate.

15. The method according to claim 14, wherein the label of the secondary antibody is selected from a group consisting of Q dot (Quantum dot), HRP (Horseradish peroxidase), alkaline phosphatase, glucose oxidase, luciferase, β-D-galactosidase, MDH (malate dehydrogenase), acetylcholinesterase, colloidal gold, fluorescein, radioactive materials and dye.

16. The method according to claim 14, wherein the label of the secondary antibody is Q dot (Quantum dot).

17. The method according to claim 14, wherein the chromogenic substrate is selected from a group consisting of DAB (diaminobenzidine), AEC(3-amino-9-ethylcarbasole), BCIP/NBT (5-bromo-4-chloro-3-indolyl-phosphate/nitroblue tetrazolium), BCIP/INT (5-bromo-4-chloro-3-indolyl phosphate/iodonitrotetrazolium), NF (New fuchsin), FRT (Fast Red TR Salt), TMB (3,3′,5,5′-tetramethyl benzidine), ABTS [2,2′-azino-bis(3-ethylbenzothiazoline-6-sulfonic acid)] and OPD (o-phenylenediamine).

18. A pharmaceutical composition for diagnosing cancer, comprising Herceptin, by detecting Herceptin-sensitive, HER2-overexpressing cells.

19. The pharmaceutical composition according to claim 18, further comprising a pharmaceutically acceptable carrier or excipient.
